(19) United States
(12) Patent Application Publication
(10) Pub. No.: US 2005/0048127 A1
(43) Pub. Date: Mar. 3, 2005

(54) Small spherical particles of low molecular weight organic molecules and methods of preparation and use thereof

(76) Inventors: Larry Brown, Newton, MA (US); Debra Lafreniere, Dighton, MA (US); John K. McGeehan, Woodbury, NJ (US)

Correspondence Address: EVEREST INTELLECTUAL PROPERTY LAW GROUP, P. O. BOX 708, NORTHBROOK, IL, 60065, US

(21) Appl. No.: 10/896,326

(22) Filed: Jul. 21, 2004

Related U.S. Application Data
(60) Provisional application No. 60/489,292, filed on Jul. 22, 2003.
(60) Provisional application No. 60/540,594, filed on Jan. 30, 2004.
(60) Provisional application No. 60/576,918, filed on Jun. 4, 2004.

(51) Int. Cl. A61K 9/00; A61K 9/14

(52) U.S. Cl. 424/489

(57) ABSTRACT

The invention provides homogeneous small spherical particles of low molecular weight organic molecules, said small spherical particles having a uniform shape, a narrow size distribution and average diameter of 0.01-200 μm. The invention further provides methods of preparation and methods of use of the small spherical particles. These small spherical particles are suitable for applications that require delivery of micron-size or nanosized particles with uniform size and good aerodynamic or flow characteristics. Pulmonary, intravenous, and other means of administration are among the delivery routes that may benefit from these small spherical particles.

### CROSS-REFERENCE TO RELATED APPLICATION:

This application claims priority to provisional application Ser. No. 60/489,292 filed on Jul. 22, 2003, provisional application Ser. No. 60/540,594 filed on Jan. 30, 2004 and provisional application Ser. No. 60/576,918 filed on Jun. 4, 2004, each of which are incorporated herein in their entirety by reference and made a part hereof.

### FEDERALLY SPONSORED RESEARCH OR DEVELOPMENT

Not Applicable.

### BACKGROUND OF THE INVENTION

1. Technical Field

The present invention provides homogeneous small spherical particles of low molecular weight active agents. These small spherical particles are, in one preferred form of the invention, characterized by a substantially uniform spherical shape, an average diameter of 0.01-200 μm, and a narrow size distribution. These small spherical particles are potentially advantageous for applications for example that require delivery of micron-sized or nano-sized particles with uniform size and good aerodynamic or flow characteristics. Pulmonary, intravenous, and other means of administration are among the delivery routes that may benefit from these small spherical particles.

2. Background Art

There is an increasing number of organic compounds being formulated for therapeutic or diagnostic effects that are poorly soluble or insoluble in aqueous solutions. Such drugs provide challenges to delivery by various routes of administration. Compounds that are insoluble in water can have significant benefits when formulated as a stable suspension of particles. Control of particle size is essential for safe and efficacious use of these formulations. Particles must be less than seven microns in diameter to safely pass through capillaries without causing emboli (Allen et al., 1987; Davis and Taube, 1978; Schroeder et al., 1978; Yokel et al., 1981). One solution to this problem is the production of small particles of the insoluble drug candidate and the creation of a small particle suspension. In this way, drugs that were previously unable to be formulated in an aqueous based system can be made suitable for intravenous administration. Particles suitable for intravenous administration will have a particle size of <7 μm, low toxicity (as from toxic formulation components or residual solvents), low excipient content, and the preservation of the bioavailability of the active agent after processing into the particle form. The current invention can lead to crystalline forms (polymorphs) that have higher rates of dissolution. It also can result in particles that have a high surface area to volume ratio and therefore can have higher rates of dissolution. Preparations of small particles of water insoluble drugs may also be suitable for oral, pulmonary, topical, ophthalmic, nasal, buccal, rectal, vaginal, transdermal, ocular, intraocular, otic, or other routes of administration.

Current approaches to increasing solubility of low molecular-weight, hydrophobic agents focus on enlargement of the surface area of the formulated particles primarily using micronization techniques, which increase the surface area to volume ratio by reducing the average particle size of the particles.

Agglomeration of micronized particles is a well-known limitation of the technique for both liquid and powder formulations.

Non-invasive delivery of drugs by the pulmonary route of administration has an important role in the treatment of respiratory diseases and other diseases. The pulmonary route offers several distinct advantages, among them the avoidance of first pass metabolism or degradation in the gastrointestinal tract, and access to a high concentration of narrow blood vessels with large surface area available for transport. This large surface area provides rapid systemic absorption when compared with the oral route of administration.

Compared with other delivery routes, pulmonary delivery offers high levels of patient compliance. It is generally regarded to be superior to the implantable and injectable administration routes and is comparable to the nasal, transdermal, and transmucosal routes. In an effort to increase patient compliance, pulmonary formulations of newer and older drugs that were only available in injectable form are being developed for the treatment of serious diseases such as diabetes mellitus.

Pulmonary delivery also offers site directed delivery of the drug to the disease site for respiratory diseases such as asthma, rhinitis, chronic obstructive pulmonary disease (COPD), cystic fibrosis (CF) and emphysema. Site directed delivery allows the most effective use of the drug, and is particularly desirable when the bioavailability of the drug is limited. Direct delivery of the drug to the disease site can potentially reduce toxicity, because the highest concentration of the drug reaches its target rather than being distributed throughout the body.

Due to these unique characteristics, the pulmonary route is suitable for both systemic and topical drug delivery and is an enabling route for the delivery of proteins and peptides. In recent years, drugs such as insulin and human growth hormone (hGH) which were previously available only as injectables have been formulated in solid dosage forms for pulmonary delivery and are currently at advanced stages of clinical trials.

The first pulmonary drugs developed were small molecule based therapies for the treatment of diseases like asthma and rhinitis. Corticosteroids that have similar structures to the naturally-produced cortisol were found to have potent anti-inflammatory action. Pulmonary formulations of corticosteroids such as beclomethasone dipropionate, budesonide, and fluticasone propionate were developed and have become a popular form of therapy for respiratory diseases that are associated with inflammation of the lungs.

Advances in pharmaceutical research have led to the development of new formulations of existing drugs to treat diseases by the pulmonary route. For instance, TOBI® (Chiron Corporation, Emeryville, Calif.) a pulmonary tobramycin solution for the treatment of cystic fibrosis, has been developed as a nebulized dosage form that can be delivered directly to the site of infection in the lungs, and is preservative-free.

Although pulmonary delivery of organic small molecules such as steroids and beta-agonists has been practiced since the invention of the first metered dose inhaler in the 1950's, most efforts have been directed toward the discovery of new therapeutic agents and the development of novel inhaler devices. Historically, little attention has been focused on the development of formulations with optimal aerodynamic characteristics; therefore, current formulations suffer from several disadvantages, including particles with broad particle size distributions, an average particle size that is larger or smaller than required and agglomerated particles. The development of compositions of small molecules with a particle size precisely in the desired range and with narrow particle size distribution is highly desirable.

Pulmonary formulations are delivered by specific types of inhaler devices. The most popular devices are the metered dose inhaler (MDI), the dry powder inhaler (DPI) and the nebulizer (US Food and Drug Administration, Center for Drug Evaluation and Research, 1998). An MDI may be used to deliver a solution or a suspension of the drug with the aid of a propellant such as CFC or HFA. The activation of MDIs and DPIs often require patient motor skill as well as respiratory coordination, which may reduce the effectiveness of the delivery. A DPI may be used to deliver a dry powder of the drug, and a nebulizer usually delivers an aqueous aerosol form of the drug. Nebulizers generally require little patient inspiratory effort in their operation. Nebulizers tend to be large, and are mainly used by children or the elderly, whose inspiratory flow rate is limited. These human factors, combined with unoptimized formulations, result in only a small fraction of the delivered dose reaching the targeted area in the lungs. Most of the dosage is typically lodged in the throat and in the mouth, and does not reach the desired location, whether it is the upper airways or the deep airways.

In a radioactive labeled study of the deposition of salbutamol in the lungs, Melchor et al. (1993) reported 20-21% deposition with an MDI and only 12% deposition with a DPI. This is particularly undesirable for drugs that are given chronically, since large quantities of the drug are continuously deposited in non-targeted areas, mainly in the oropharynx. High oropharyngeal deposition can have adverse local effects, such as oral thrush or candiasis. Because the risk of adverse effects resulting from chronic use of corticosteroids is dose dependent, a reduction in the delivered dose is predicted to lower the risk of side effects (Corren et al., 2003). A dry powder of the drug with particles at the desired size range and a narrow particle size distribution can result in reduced dosing, because the portion of the drug that reaches its destination is increased, therefore the administered dose can be minimized. This has been demonstrated for fluticasone, budesonide, and beclomethasone by Corren et al. (ibid).

Conventional pulmonary formulations are the direct result of pharmaceutical cGMP manufacturing processes that typically have several stages. One of the final stages in many pharmaceutical processes is crystallization, which serves as a purification step, and as a method to precipitate solid out of solution. Current crystallization techniques lead to particles with various shapes and sizes, and most resulting powders have particles that are much larger than that required for pulmonary delivery. In addition, many active pharmaceutical agents are hydrophobic agents with limited solubility and hence limited bioavailability. Reduction of particle size lowers the energy barrier required for dissolution. Thus the size of the particles can be reduced and this is often attained by adding a physical grinding or micronization step during or post crystallization.

For example, U.S. Pat. No. 5,314,506 to Midler et al. describes a method to decrease particle size by the addition of an impinging jet step prior to the crystallization stage.

Precipitation from solution using an antisolvent system is a one of the most common crystallization methods (Wey et al. 2001). In this type of crystallization system a solute is crystallized from a primary solvent by the addition of a second solvent (antisolvent) in which the solute is relatively insoluble. A solution of the solute in a solvent, which is often saturated or close to saturation, is initially formed. Then, an antisolvent that is miscible with the primary solvent is added. The antisolvent is selected such that the solute is relatively insoluble in the antisolvent. When the antisolvent is added to the solution, the solute precipitates out of the binary mixture due to the reduction in solubility of the solute in the binary mixture compared with the solvent.

### SUMMARY OF THE INVENTION

The small spherical particles described herein have a uniform size, preferably in the range of 0.1-4 microns, and have a substantially uniform spherical shape. These particles have a higher ratio of surface area to volume, a reduced tendency to agglomerate compared with conventional micronized particles, and a uniform aerodynamic shape. An increase in the surface area of a formulated compound may enhance the dissolution rate of the drug.

Further disclosed herein are methods for preparing homogeneous small spherical particles comprising low molecular weight agents. These methods offer several advantages including low processing temperatures, formation of small spherical particles in a desired size range, with a narrow size distribution and batch-to-batch uniformity. These methods result in high yields when compared with conventional micronization techniques, and provide for recovery of substantially all of the starting material in the desired size range. These methods do not require a separate and time consuming step of sieving to remove oversized particles.

Since the small spherical particles are substantially of the same size and shape, batch-to-batch uniformity can be achieved. Additionally, these processes can significantly reduce fabrication time and costs, when compared with conventional processes. The small spherical particles described herein are particularly suitable, for example, for targeted delivery to the lungs. For pulmonary delivery, the particles generally should have an MMAD of 5 μm or less, depending on the area of the lung targeted for treatment (i.e., deep lung, whole lung, etc.). The small spherical particles can be formed in a size range that is suitable for deposition in specific areas of the lungs. Diseases of the pulmonary airways, such as asthma, COPD, emphysema, and others, can be characterized by the area of the lung that is affected by the disease. Asthma is considered a disease of the entire lung, with inflammation of the central airways as well as the periphery of the lungs (Corren et al., 2003). It is known that in order to reach the lung periphery, the drug's aerodynamic particle size should be 0.5 to 3.0 microns (Brown, 2002). This allows targeted delivery of the drug to the alveoli. Furthermore, systemic delivery through the lungs generally requires that the drug be delivered to the periphery of the lungs, i.e., the alveoli. The small spherical particles described herein can be produced in a size range that allows effective deposition at the disease site, and since they are of substantially the same size, a high efficiency of medication delivery to the desired lung location.

These and other aspects and attributes of the present invention will be discussed with reference to the following drawings and accompanying specification.

### BRIEF DESCRIPTION OF THE DRAWINGS

FIG. 1 is a scanning electron microscopy (SEM) image of micronized beclomethasone dipropionate (BDP), which is used as a starting material in the process described in example 1. This image presents the characteristics of many micronized low molecular weight drugs. The BDP particle size varies between hundreds of nanometers to 50 microns. The particle size distribution is broad and the particles have random shapes.

FIG. 2 depicts small spherical particles of beclomethasone dipropionate (BDP) prepared according to the method described in Example 1 below. These small spherical particles are characterized by a uniform shape, an average particle size of 2 microns, and an extremely narrow size distribution. The small spherical particles are substantially spherical, and are substantially the same size.

FIG. 3 presents X-Ray Powder Diffraction patterns (XRPD) of micronized beclomethasone dipropionate starting material (bottom), and XRPD patterns of two batches of BDP small spherical particles fabricated according to example 1, below.

FIG. 4 is an SEM image of micronized budesonide, which is used as the starting material in example 2. The budesonide particles size ranges between hundreds of nanometers to 100 microns. Particle size distribution is broad, and the particles have random shapes.

FIG. 5 depicts small spherical particles of budesonide prepared according to the method described in example 2, below. These small spherical particles are characterized by a uniform shape, an average particle size of 2 microns, and an extremely narrow size distribution. The small spherical particles are substantially spherical, and are substantially of the same size.

FIG. 6 presents a XRPD of micronized budesonide starting material (top), and XRPD patterns of small spherical particles of budesonide (bottom) fabricated according to example 2, below.

FIG. 7 presents the aerodynamic particle size distribution (PSD) of budesonide small spherical particles measured by an Aerosizer. The distribution is calculated based on time- of-flight.

FIG. 8 is an SEM image of micronized itraconazole, which is used as the starting material in example 3. The itraconazole particle size ranges between hundreds of nanometers to microns. Particle size distribution is broad, and the particles have random shapes.

FIG. 9 depicts small spherical particles of itraconazole prepared according to the method described in example 3, below. These small spherical particles are characterized by a uniform shape, an average particle size of 1 micron, and an extremely narrow size distribution. The small spherical particles are substantially spherical, and are substantially of the same size.

FIG. 10 depicts the particle size distribution of itraconazole microspheres by light scattering. The small spherical particles were suspended in deionized water with a surfactant.

FIG. 11 is a schematic flow diagram summarizing the process of making small spherical particles of beclamethasone dipropionate (BDP).

FIG. 12 is a schematic diagram of an apparatus for preparing small spherical particles.

FIG. 13 is a schematic end view of an apparatus for preparing small spherical particles.

FIG. 14 is a schematic view of an apparatus for preparing small spherical particles.

### DETAILED DESCRIPTION OF THE PREFERRED EMBODIMENT

While this invention can have embodiments in many different forms, the principles shown in the drawings, and that will be described herein in detail, has specific embodiments thereof with the understanding that the present disclosure is to be considered as an exemplification of the principles of the invention and is not intended to limit the invention to the specific embodiments illustrated.

The Particles

The small spherical particles of the present invention preferably have an average particle size of from about 0.01 μm to about 200 μm, more preferably from about 0.1 μm to about 10 μm and most preferably 0.1 μm to about 4 μm, as measured by dynamic light scattering methods, e.g., photocorrelation spectroscopy, laser diffraction, low-angle laser light scattering (LALLS), medium-angle laser light scattering (MALLS), or by light obscuration methods (Coulter method, for example), or other methods, such as rheology, or microscopy (light or electron). Particles for pulmonary delivery will have an aerodynamic particle size determined by time of flight measurement by a TSI Corporation Aerosizer or Andersen Cascade Impactor.

The small spherical particles are substantially spherical. What is meant by substantially spherical is that the ratio of the lengths across perpendicular axes of the particle cross-section is from 0.5 to 2.0, more preferably from 0.8 to 1.2 and most preferably from 0.9 to 1.1.

Surface contact is minimized between and among substantially spherical particles which minimizes the undesirable agglomeration of the particles. Faceted shapes and flakes have flat surfaces that present an opportunity for large contact areas between adjacent particles. For particles having a broad size distribution where there are both relatively large and relatively small particles, smaller particles can fill in the gaps between the larger particles, thereby creating new contact surfaces.

Typically, small spherical particles made by the process in this invention are substantially non-porous and have a density greater than 0.50/cm³, more preferably greater than 0.750/cm³ and most preferably greater than about 0.85/cm³. A preferred range for the density is from about 0.50 to about 2.00 g/cm³ and more preferably from about 0.75 to about 1.750 g/cm³ and even more preferably from about 0.85 g/cm³ to about 1.50 g/cm³. This is in contrast to pulmonary, low density particles produced by spray drying that are typically produced at approximately 0.4 g/cm³. The higher density particles allow for greater quantities of the active agent to be delivered to the patient compared with lower density particles. It is a particularly desirable feature for drugs that are not very potent, thus larger quantities of the drug can be delivered, or for drugs that are given chronically, a decrease in the dosage size can decrease adverse effects and increase patient compliance.

The small spherical particles can have a smooth surface profile or a textured surface profile. A smooth surface profile is generally smooth, which means the distance from any point on the surface of the particle to the center of the particle is the same distance. Textured surfaces is meant to refer to surface variations having dimensions that are far smaller than the overall diameter of the particle. The textured surface can take many forms including regularly spaced or irregularly spaced proturberances or indentations in the particle surface, longitudinally or latitudinally extending lines or grooves or cracks or other surface disruption, or other forms or combinations of surface irregularities that can occur on a drug particle. The texturing on a particle surface can be located over a single portion of the surface or on multiple portions of the surface of the particle or over substantially the entire surface of the particle.

The spherical shape of the small spherical particles combined with their uniform size provide a unique composition where the particles are spheres of uniform size, which by definition is the physical form with the least amount of surface contact. It is well known that interactions between particles along surface contact areas, such as electrostatic, van der Waals and others, strongly depend on the distance between adjacent particles. Thus, a reduction in the contact area between particles decreases the interparticle attractive forces and can lead to particles with a significantly reduced tendency for agglomeration. Reduced interparticle attraction between the small spherical particles results in powders with improved flowability, and when in suspensions, show reduced tendency to agglomerate. Compared to traditional powders of micronized drugs, the small spherical particles disclosed herein have a reduced tendency to agglomerate, sediment or flocculate.

The particles also preferably have substantially the same particle size. Particles having a broad size distribution where there are both relatively big and small particles allow for the smaller particles to fill in the gaps between the larger particles, thereby creating new contact surfaces. A broad size distribution can result in the creation of many contact opportunities for binding agglomeration. This invention creates spherical particles with a narrow size distribution, thereby minimizing opportunities for contact agglomeration. What is meant by a narrow size distribution is a preferred particle size distribution would have a ratio of the diameter of the 90^{th} percentile of the small spherical particles to the diameter of the 10^{th} percentile less than or equal to 5. More preferably, the particle size distribution would have ratio of the diameter of the 90^{th} percentile of the small spherical particles to the diameter of the 10^{th} percentile less than or equal to 3. Most preferably, the particle size distribution would have ratio of the diameter of the 90^{th} percentile of the small spherical particles to the diameter of the 10^{th} percentile less than or equal to 2.

Geometric Standard Deviation (GSD) can also be used to indicate the narrow size distribution. GSD calculations involve determination of the effective cutoff diameter (ECD) at the cumulative mass less than percentages of 15.9% and 84.1%. GSD is equal to the square root of the ratio of the ECD cumulative mass less than 84.17% to ECD cumulative mass less then 15.9%. The GSD has a narrow size distribution when GSD<2.5, more preferably less than 1.8.

The small spherical particles are preferably nearly 100% active agent or a combination or blend of active agents that are substantially free of any excipients. What is meant by “substantially free of excipients” is that the active agent or active agents is present from about 70% to less than 100% by weight of the small spherical particles, excluding water. More preferably, the active agent(s) is greater than about 90% by weight of small spherical particles and most preferably the small spherical particles will have 95% or greater by weight of the active agent. These ranges, as well as all other ranges recited herein, shall include any range, sub-range, or combination of ranges therein.

In some instances it may be desirable for the particle to include an optional bulking agent or other surfactant provided these additives do not substantially impact the effectiveness of the agent. Bulking agents can include saccharides, disaccharides, polysaccharides and carbohydrates.

The small spherical particles can be crystalline, semi-crystalline, or non-crystalline.

The Active Agent

The active agent of the present invention is a low molecular weight organic substance. A low molecular weight substance is one having a molecular weight of equal to or less than approximately 1,500 Daltons. As set forth above, the particles can have a single active agent or more than one active agent.

The active agent can be hydrophobic or hydrophilic. In a preferred embodiment, the active agent is a sparingly water soluble compound. What is meant by sparingly water soluble is that the active agent has a solubility in water of less than 10 mg/mL, preferably less than 1 mg/mL.

The active agent of the present invention is preferably a pharmaceutically active agent, which can be a therapeutic agent, a diagnostic agent, a cosmetic, a nutritional supplement, or a pesticide.

Examples of an active agent suitable for the present invention include but are not limited to steroids, beta-agonists, anti-microbials, antifungals, taxanes (antimitotic and antimicrotubule agents), amino acids, aliphatic compounds, aromatic compounds and urea compounds.

In a preferred embodiment, the active agent is a therapeutic agent for treatment of pulmonary disorders. Examples of such agents include steroids, beta-agonists, anti-fungal, and anti-microbial compounds. Examples of steroids include but are not limited to beclomethasone (including beclomethasone dipropionate), fluticasone (including fluticasone propionate), budesonide, estradiol, fludrocortisone, flucinonide, triamcinolone (including triamcinolone acetonide), and flunisolide. Examples of beta-agonists include but are not limited to salmeterol xinafoate, formoterol fumarate, levo albuterol, bambuterol and tulobuterol.

Examples of anti-fungal agents include but are not limited to itraconazole, fluconazole, and amphotericin B.

Numerous combinations of active agents may be desired including, for example, a combination of a steroid and a beta-agonist, e.g., fluticasone propionate and salmeterol, budesonide and formeterol, etc.

Also included are pharmaceutically accepted salts, esters, hydrates and solvates of these compounds. Also included in the above compounds are crystalline or a crystalline polymorph or pseudo-polymorph of the small organic molecule.

The present invention further provides additional steps for altering the crystal structure of the active agent to produce the agent both in the desired size range and also in the desired crystal structure to optimize the dissolution rate of the agent. What is meant by the term crystal structure is the arrangement of the molecules within a crystal lattice. Compounds that can be crystallized into different crystal structures are said to be polymorphic. Identification of polymorphs is an important step in drug formulation since different polymorphs of the same drug can show differences in dissolution rate, therapeutic activity, bioavailabilty and suspension stability. Accordingly, it is important to ensure the polymorphic form consistency of the compound for batch-to-batch reproducibility.

In another form of the particles, the particles can include agents to vary the rate of release of the agent or to provide for targeting of the agent to a particular site for treatment.

Examples of pulmonary disorders include, but not limited to, allergy rhinitis, bronchitis, asthma, chronic obstructive pulmonary diseases (COPD), emphysema, infectious disease, and cystic fibrosis.

Optional Excipients

The system of the present invention may include one or more excipients. The excipient may imbue the active agent or the particles with additional characteristics such as increased stability of the particles or of the active agents or of the carrier agents, controlled release of the active agent from the particles, or modified permeation of the active agent through biological tissues. Suitable excipients include, but are not limited to, carbohydrates (e.g., trehalose, sucrose, mannitol), cations (e.g., Zn²⁺, Mg²⁺, Ca²⁺), anions (e.g., SO₄²⁻), amino acids (e.g., glycine), lipids, phospholipids, fatty acids, surfactants, triglycerides, bile acids or their salts (e.g., cholate or its salts, such as sodium cholate; deoxycholic acid or its salts), fatty acid esters, and polymers (e.g., amphiphilic, hydrophilic polymers, such as polyethylene glycol or lipophilic polymers).

In vivo Delivery of the Particles

The small spherical particles containing the active agent in the present invention are suitable for in vivo delivery to a subject in need of the agent by a suitable route, such as injectable, topical, oral, rectal, nasal, pulmonary, vaginal, buccal, sublingual, transdermal, transmucosal, otic, intraocular or ocular. The particles can be delivered as a stable liquid suspension, tablet, a dry powder, a powder suspended in a propellant such as CFC or HFA, or in a nebulized form.

A preferred delivery route is pulmonary delivery. In this route of delivery, the particles may be deposited to the deep lung, the central or peripheral area of the lung, or the upper respiratory tract of the subject in need of the therapeutic agent. The particles may be delivered as a dry powder by a dry powder inhaler, or they may be delivered in suspension by a metered dose inhaler or a nebulizer. When delivered by the pulmonary route, the active agent can be used to treat respiratory disorders local to the lungs of the subject, or the active agent can be absorbed into the systemic circulation for treatment of other diseases.

Another preferred route of delivery is parenteral, which includes intravenous, intramuscular, subcutaneous, intraperitoneal, intrathecal, epidural, intra-arterial, intra-articular and the like.

The Process and Aipparatus

One method for preparing the small spherical particles of the present invention include the following steps: (1) providing a solution of the active agent in a first solvent; (2) adding a second solvent to the solution to form a three component solution of the two solvents and the active agent; the solubility of the active agent in the second solvent is lower than in the first solvent (3) spreading the three-component solution on a surface to form a thin film; and (4) evaporating the solvents by passing a stream of gas over the film to form small spherical particles of the active agent on the surface, wherein the gas does not react with the active agent.

The small spherical particles are formed during the evaporation step, which also cools the thin film to facilitate the formation of the small spherical particles. It is preferred that the steps are carried out at or below ambient temperature of about 25° C. Any or all of the solvents, the gas, the agent and pertinent portions of the apparatus used to make the particles may be cooled in order to facilitate particle formation and removal from the surface. The method can also include additional steps of drying the small spherical particles on the surface, removing the small spherical particles from the surface, and forming a dry powder of the small spherical particles.

The first solvent can be an organic solvent or an aqueous medium, depending on the active agent. Suitable organic solvents include but are not limited to N-methyl-2-pyrrolidinone (N-methyl-2-pyrrolidone), 2-pyrrolidinone (2-pyrrolidone), 1,3-dimethyl-2-imidazolidinone (DMI), dimethylsulfoxide, dimethylacetamide, volatile ketones such as acetone, methyl ethyl ketone, acetic acid, lactic acid, acetonitrile, methanol, ethanol, isopropanol, 3-pentanol, n-propanol, benzyl alcohol, glycerol, tetrahydrofuran (THF), polyethylene glycol (PEG), PEG-4, PEG-8, PEG-9, PEG-12, PEG-14, PEG-16, PEG-120, PEG-75, PEG-150, polyethylene glycol esters, PEG-4 dilaurate, PEG-20 dilaurate, PEG-6 isostearate, PEG-8 palmitostearate, PEG-150 palmitostearate, polyethylene glycol sorbitans, PEG-20 sorbitan isostearate, polyethylene glycol monoalkyl ethers, PEG-3 dimethyl ether, PEG-4 dimethyl ether, polypropylene glycol (PPG), polypropylene alginate, PPG-10 butanediol, PPG-10 methyl glucose ether, PPG-20 methyl glucose ether, PPG-15 stearyl ether, propylene glycol dicaprylate/dicaprate, propylene glycol laurate, and glycofurol (tetrahydrofurfuryl alcohol polyethylene glycol ether), propane, butane, pentane, hexane, heptane, octane, nonane, decane, or a combination thereof.

In a preferred embodiment in which the active agent is a hydrophobic compound, the first solvent is an aqueous-miscible organic solvent, for example, an alcohol such as ethanol, and the second solvent is an aqueous medium. The three-component system therefore comprises the hydrophobic active compound, ethanol and water.

The first solvent or the second solvent or both the first solvent and the second solvent are preferably a volatile solvent. What is meant by volatile is that its vapor pressure is higher than that of water. In a preferred embodiment, the first solvent is more volatile than the second solvent, e.g., ethanol is the first solvent and water is the second.

In one process of the present invention, the step of providing the solution of the active agent in the first solvent includes the steps of adding the active agent to the first solvent and sonicating the first solvent to completely dissolve the agent in the first solvent.

In one process of the present invention, the step of spreading the mixture on a surface to form a thin film includes the steps of transferring the mixture to a rotary evaporating flask and slowly rotating the flask to coat the mixture on the surface of the flask.

The gas used to evaporate the solvent from the thin film of the solution is preferably inert but can be noninert. Examples of suitable gases that can be used to evaporate the solvents from the thin film of the solution include but are not limited to nitrogen, hydrogen and noble gases such as helium and argon. The flow rate of the gas should be optimized according to the active agent, first solvent and/or the second solvent used in the process. The gas inflow can be stopped once the solvents are completely evaporated. Optionally, the gas inflow can continue at a reduced flow rate for a short period of time (e.g., about 3 minutes) to dry the small spherical particles on the surface.

The method can also include additional steps of removing the small spherical particles from the surface and forming dry powder of the small spherical particles. In one embodiment, the steps of removing the small spherical particles from the surface include adding a minimal amount of the second solvent to remove the small spherical particles from the surface. Preferably, the second solvent is ice-cold water at about 4° C. Optionally, the second solvent can be sonicated, preferably on ice, to facilitate the removal process. The second solvent can also be further removed to form a dry powder by a process such as freeze-drying or lyophilization.

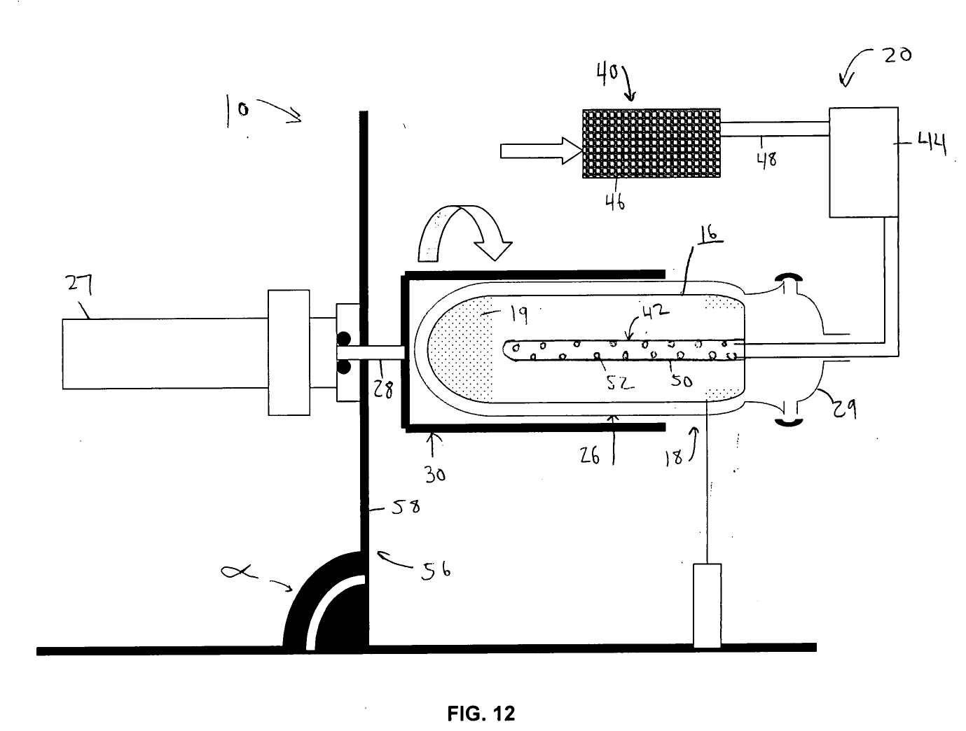

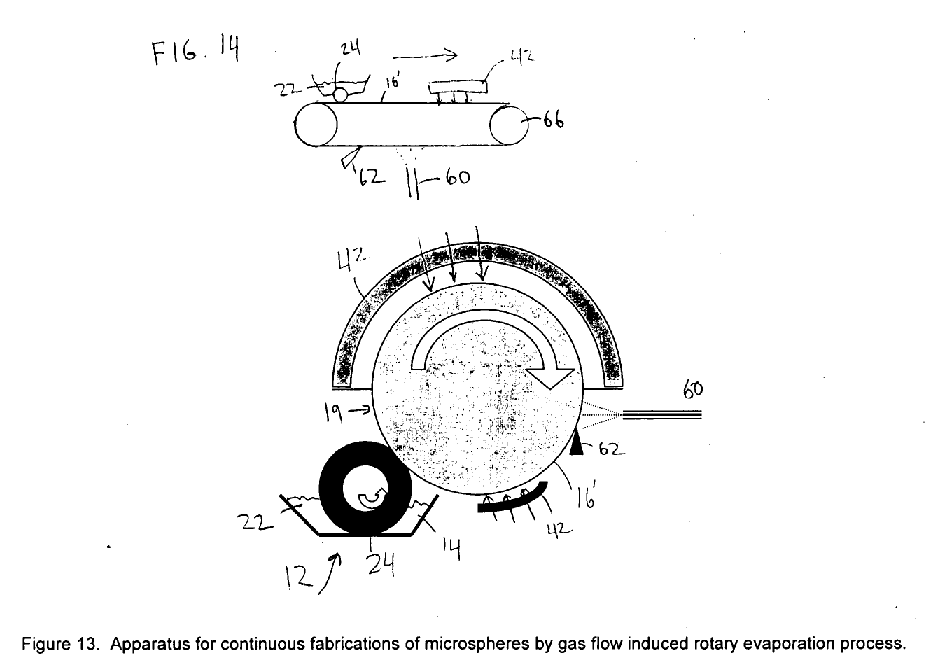

FIGS. 12 and 13 show an apparatus suitable for this process which includes a fluid delivery device or system 12 (FIG. 13) for delivering the three-component solution from a source 14 to a surface 16, a motive device 18 for moving the surface with respect to the source 14 to form a thin film 19 of the three-component solution on the surface 16, and a gas delivery device or system 20 for supplying gas under pressure to the surface 16 or the film 19 or both.

In a process for continuously preparing the particles described herein, the fluid delivery device includes the source 14 having a quantity of the solution 22, a device 24 for supplying the solution to the surface 16, and, in this case, is a transfer roller. The transfer roller 24 is mounted for rotation about an axis and has an outer circumferential portion placed in contact with the solution which is then carried on an outer circumferential portion of the roller into engagement with the surface 16 to form a thin film 19 of the solution on the surface 16. It is contemplated that the delivery device 24 can take on many forms and include numerous different types of applicators, such as spray applicators or other type applicator, as long as the applicator is capable of depositing the solution in a controlled fashion onto the surface 16 to form a thin film 19 thereon.

In a batch process, the solution can be added to the reaction vessel using standard laboratory techniques, such as pipetting or other techniques well known in the art.

The surface 16 can have various cross-sectional shapes including flat, curved, round, elliptical, undulating or irregular. As shown in FIGS. 12 and 13, in one preferred form of the invention, the surface is curved and preferably is generally cylindrical 26. It is contemplated that curved surfaces could also be, conical, frusto-conical, or spherical. As shown in FIGS. 12 and 13, the surface 16 is carried on an internal 16 or external surface 16′ of the glass cylinder 26. The glass cylinder, in a preferred form, is a 10 liter glass reactor vessel with an optional glass reactor head 29, which may be clamped to seal the vessel.

The surface 16 can have a smooth profile, having a substantially constant height dimension across the surface, or the surface can be textured either to decrease the contact angle of the solution on the surface or to increase the wettability of the solution on the surface. Textured surfaces include those that have a surface profile that does not have a constant height for every point along the surface. Textured surfaces include but are not limited to a matte surface, frosted, embossed, or the like. In a preferred form of the invention, the surface is a smooth surface.

Suitable surfaces are made from a material such as a polymer, metal, ceramic, or glass. The material can be rigid, semi-rigid or flexible. What is meant by flexible is having a modulus of elasticity of less than 20,000 psi. What is meant by rigid is having a modulus of elasticity of greater than 40,000 psi. Semi-rigid materials have a modulus of elasticity between 20,000 psi and 40,000 psi. In a most preferred form of the invention, the surface is glass.

Suitable polymers to form the surface include those that do not react with the active agent and include polyolefins, cyclic olefins, bridged polycyclic hydrocarbons, polyamides, polyesters, polyethers, polyimides, polycarbonates, polystyrene, polyvinyl chloride, ABS, polytetrafluoroethylene (PTFE), styrene and hydrocarbon copolymers, synthetic rubbers and the like. The term polyolefin used herein is meant to include homopolymers and copolymers of ethylene, propylene, butene, pentene, hexene, heptene, octene, nonenene, and decene. Suitable copolymers of ethylene include: (a) ethylene copolymerized with monomers selected from the group of α-olefins having 3-10 carbons, lower alkyl and lower alkene substituted carboxylic acids and ester and anhydride derivatives thereof, (b) ethylene propylene rubbers, (c) EPDM, (d) ethylene vinyl alcohol, and (e) ionomers. Preferably, the carboxylic acids have from 3-10 carbons. Such carboxylic acids, therefore, include acetic acid, acrylic acid, and butyric acid. Suitable acrylic acid containing polymers include PMMA, sold under the trade name Plexiglas. The term lower alkene and lower alkyl is meant to include a carbon chain having from 2-18 carbons, more preferably 2-10 and most preferably 2-8 carbons. Thus, a subset of this group of comonomers includes, as a representative but non-limiting example, vinyl acetates, vinyl acrylates, methyl acrylates, methyl methacrylates, acrylic acids, methacrylic acids, ethyl acrylates, and ethyl acrylic acids.

Suitable homopolymer and copolymers of cyclic olefins, bridged polycyclic hydrocarbons, and blends thereof can be found in U.S. Pat. Nos. 4,874,808; 5,003,019; 5,008,356; 5,288,560; 5,218,049; 5,854,349; 5,863,986; 5,795,945; and 5,792,824, which are incorporated in their entirety herein by reference and made a part hereof. In a preferred form of the invention, these homopolymers, copolymers, and polymer blends will have a glass transition temperature of greater than 50° C., more preferably from about 70° C. to about 180° C., a density greater than 0.910 g/cc, more preferably from 0.910 g/cc to about 1.3 g/cc and most preferably from 0.980 g/cc to about 1.3 g/cc, and have from at least about 20 mole % of a cyclic aliphatic or a bridged polycyclic in the backbone of the polymer, more preferably from about 30-65 mole % and most preferably from about 30-60 mole %.

In a preferred form of the invention, suitable cyclic olefin monomers are monocyclic compounds having from 5 to about 10 carbons in the ring. The cyclic olefins can be selected from the group consisting of substituted and unsubstituted cyclopentene, cyclopentadiene, cyclohexene, cyclohexadiene, cycloheptene, cycloheptadiene, cyclooctene, and cyclooctadiene. Suitable substituents include lower alkyl, acrylate derivatives and the like.

In a preferred form of the invention, suitable bridged polycyclic hydrocarbon monomers have two or more rings and more preferably contain at least 7 carbons. The rings can be substituted or unsubstituted. Suitable substitutes include lower alkyl, aryl, aralkyl, vinyl, allyloxy, (meth) acryloxy and the like. The bridged polycyclic hydrocarbons are selected from the group consisting of those disclosed in the above incorporated patents and patent applications. A most preferred polycyclic hydrocarbon is a norbornene homopolymer or a norbornene copolymer with ethylene. Suitable norbornene containing polymers are sold by Ticona under the tradename TOPAS, by Nippon Zeon under the tradename ZEONEX and ZEONOR, by Daikyo Gomu Seiko under the tradename CZ resin, and by Mitsui Petrochemical Company under the tradename APEL.

The polymeric material can be formed into the surface by extrusion, coextrusion, lamination, extrusion lamination, injection molding, blow molding, thermoforming, or other processing technique. The material can be a flexible, semiflexible or rigid. The material can be a monolayer film or a multiple layer film. The film can have a protein compatible surface, such as the films disclosed in U.S. Pat. No. 6,309,723 which is incorporated in its entirety herein by reference and made part herein. The material can also be fabricated into numerous shapes and sizes as desired.

Suitable metals include aluminum, stainless steel, vanadium, platinum, titanium, gold, beryllium, copper, molybdenum, osmium, nickel, or other suitable alloys or metals or metal composites.

Suitable ceramics include Cordierite, Albite (Feldspar NaAlSi₃O₈), Augite (Iron-Magnesium Silicate), Biotite K (Mg,Fe)₃-(AlSi₃O₁₀)(OH)₂, Hornblende (Iron-Magnesium Silicate), Illite KAl₂(AlSi₃O₁₀)-(OH)₂, Kaolinite (Al₂O₃—2SiO₂—4H₂O), Labradorite (Feldspar; 60% CaAl₂Si₂O₈+40% NaAlSi₃O₈), Montmorillonite Al₂O₃—4SiO₂-nH₂O, Muscovite (KAl₂(AlSi₃O₁₀)-(OH)₂), Orthoclase (Feldspar KAlSi₃O₈), Quartz (SiO₂), Mica (KAL₂(ALSi₃O₁₀)(OH)₂), Mica (K(Mg,Fe)₃(AlSi₃O₁₀)(OH)₂), Amphibole ((Ca—Na)₂₋₃ (Mg,Fe,Al)₅Si₆(SiAl)₂O₂₂(OH)₂), Amphibole (CaMg₅Si₈O₂₂(OH)₂), Pyroxene (X₂Si₂O₆), Olivine ((Mg, Fe)₂SiO₄), Chlorates ((Mg,Fe,Al)₆(Al,Si)₄O₁₀(OH)₈), Feldspar (K₂O Al₂O₃ 6SiO₂), Feldspar (Na₂O Al₂O₃6SiO₂,CaO Al₂O₃2SiO₂), Mullite, 3Al₂O₃—2SiO₂, K0.5Na0.5NbO3, Fused Quartz, Fused Quartz, Steatite (Magnesium Silicon Oxide), Vermiculite, Magnesium Aluminum Iron Silicate, Silica Aerogel, AREMCO Aremcolox™ 502-1100, Unfired, AREMCO Aremcolox™ 502-1100, Full-fired, AREMCO 618 Cerama-bond™, AREMCO 677 Pyro-Putty®, AREMCO 685 Cerama-bond™, AREMCO Cerama-cast™ 645N, AREMCO Cerama-cast™ 646, AREMCO Cerama-Fab™ 665, AREMCO Cerama-cast™ 674, AREMCO Cerama-bond™ 3062, AREMCO Cerama-Dip™ 538N, CeramTec Grade 645 Steatite (MgO—SiO₂), CeramTec Grade 665 Steatite (MgO—SiO₂), CeramTec Grade 447 Cordierite (2MgO—2Al₂O₃—5SiO₂), CeramTec Grade 547 Cordierite (2MgO—2Al₂O₃—5SiO₂), CeramTec Grade 701 Cordierite (2MgO—2Al₂O₃—5SiO₂), Steatite (Magnesium Silicon Oxide), Vermiculite, Magnesium Aluminum Iron Silicate, Magnesium Oxide (MgO) Single Crystal Substrate, Spinel (MgAl204) Single Crystal Substrate, AREMCO 571 Cerama-bond™, AREMCO Cerama-cast™ 583, AREMCO Cerama-cast™ 584, AREMCO Cerama-cast™ 672, CeramTec Grade 645 Steatite (MgO—SiO₂), CeramTec Grade 665 Steatite (MgO—SiO₂), CeramTec Grade 447 Cordierite (2MgO—2Al₂O₃—5SiO₂), CeramTec Grade 547 Cordierite (2MgO—2Al₂O₃—5SiO₂), CeramTec Grade 701 Cordierite (2MgO—2Al₂O₃13 5SiO₂), Du-Co DC-9-L-3 Steatite, Du-Co DC-10-L-3 Steatite, Du-Co DC-16-L-3 Steatite, Du-Co CS-144-L-5 Steatite, Du-Co DC-200-L-5 Fosterite, Du-Co DC-187 Magnesium Oxide, EDO Ceramics EC-98 Lead Magnesium Niobate Piezoelectric, GBC L3 Steatite, ICE Steatite L-4, ICE Steatite L-5, LUMINEX® Magnesia, Steatite (Morgan Matroc), NAPCO C90 Magnesite, NAPCO C95 Magnesite, NAPCO H-98-Magnesite, NAPCO F96—Fused Magnesia, Sapco C 221 Steatite, Sapco C 220 Steatite, Sapco C 410 Steatite, Magnesium Oxide, MgO (Periclase), Magnesium Peroxide, MgO₂, 99.6% Alumina, thin-film substrate, Cordierite, Albite (Feldspar NaAlSi₃O₈), Biotite K (Mg,Fe)₃-(AlSi₃O₁₀) (OH)₂, Illite KAl₂(AlSi₃O₁₀)-(OH)₂, Kaolinite (Al₂O₃—2SiO₂—4H₂O), Labradorite (Feldspar; 60% CaAl₂Si₂O₈+40% NaAlSi₃O₈), Montmorillonite Al₂O₃—4SiO₂-nH₂O, Muscovite (KAl₂(AlSi₃O₁₀)-(OH)₂), Orthoclase (Feldspar KAlSi₃O₈), Mullite, 3Al₂O₃—2SiO₂, Germanium Mullite, 3Al₂O₃—2GeO₂, Spinel, MgAl₂O₄, AO 95 Aluminum Oxide Ceramic Substrate, 95% Purity, AO 98 Aluminum Oxide Ceramic Substrate, 98% Purity, Sapphire (Aluminum Oxide—A1203) Single Crystal, Spinel (MgAl2O4) Single Crystal Substrate, Lithium Aluminum Oxide (LiAlO2) Single Crystal Substrate, Aluminum Oxide Ceramic—Alumina 96%, Aluminum Oxide Ceramic—Alumina 97.5%, Aluminum Oxide Ceramic—Alumina 98%, Aluminum Oxide Ceramic—Alumina 99.5%, Lanthanum Aluminum Oxide (LaAlO3) Single Crystal Substrate, Thorium-Doped Lanthanum Aluminum Oxide (Th:LaAlO3) Single Crystal Substrate, Strontium Lanthanum Aluminate (SrLaAlO3) Single Crystal Substrate, Yttrium Aluminate (YAlO3) Single Crystal Substrate, Beryllia, 99.5%; BeO, Calcium Hydroxyapatite, Ca₁₀(PO₄)₆(OH)₂, Tetracalcium-Phosphate, Ca₄PO₉, Tricalcium-Phosphate (TCP), CA₃(PO₄)₂, Cordierite, Germanium Mullite, 3Al₂O₃—2GeO₂, Dy2O3, Er2O3, Yb2O3, Lithium Aluminum Oxide (LiAlO2) Single Crystal Substrate, Lithium Gallium Oxide (LiGaO2) Single Crystal Substrate, Neodymium Gallium Oxide (NdGaO3) Single Crystal Substrate, Zinc Oxide (ZnO) Single Crystal Substrate, Strontium Titanate (SrTiO3) Single Crystal Substrate, Lanthanum Aluminum Oxide (LaAlO3) Single Crystal Substrate, Thorium-Doped Lanthanum Aluminum Oxide (Th:LaAlO3) Single Crystal Substrate, Strontium Lanthanum Aluminate (SrLaAlO3) Single Crystal Substrate, Strontium Lanthanum Galate (SrLaGaO₃) Single Crystal Substrate, Yttrium Aluminate (YAlO3) Single Crystal Substrate, AREMCO Aremcolox™ 502-1550, Low Density, AREMCO Aremcolox™ 502-1550, Med. Density, AREMCO Cerama-cast™ 674, AREMCO Corr-Paint™ CP3000, AREMCO Corr-Paint™ CP3010, AREMCO Corr-Paint™ CP4000, Ceralloy 418, Beryllium Oxide, BeO, Chromium Carbide, Cr3C2, Hafnium Carbide, HfC, Molybdenum Carbide, Mo2C, Niobium Carbide, Silicon Carbide, CVD, Silicon Carbide, sintered alpha, Silicon Carbide, sublimed, Tantalum Carbide, Titanium Carbide, TiC, Vanadium Carbide, Tungsten Carbide, W2C, Tungsten Carbide, WC, Zirconium Carbide, Silicon Carbide (6H) Single Crystal Substrate, GE Advanced Ceramics Tantalum Carbide (TaC) Coating, GE Advanced Ceramics Niobium Carbide (NbC) Coating, GE Advanced Ceramics Zirconium Carbide (ZrC) Coating, AREMCO Cerama-cast™ 673, Ceralloy 546, Boron Carbide, B4C, Ceralloy 146, Silicon Carbide, SiC, Destech Silicon Carbide, Solid or Foamed, Gouda Vuurvast CURON 140 K Dense Refractory Castable, Gouda Vuurvast CURON 160 H SIC GM Dense Refractory Castable, Gouda Vuurvast VIBRON 160 H SiC Dense Vibrating Refractory Castable, Gouda Vuurvast VIBRON 160 K Dense Vibrating Refractory Castable, Gouda Vuurvast VIBRON 160 K 50 Dense Vibrating Refractory Castable, Gouda Vuurvast VIBRON 162 K Sp Dense Vibrating Refractory Castable, Magnesium Fluoride, MgF₂, (Sellaite), Bischofite (MgCl₂—6H₂O), Tachhydrite (2MgCl₂—CaCl₂—12H₂O), Reade Advanced Materials Synthetic Cryolite Powder (Na₃AlF₆ or 3NaF.AlF₃), Copper Bromide, CuBr, Cubic, Copper Bromide, CuBr, Hexagonal, Copper Chloride, CuCl, Cubic (Nantokite), Copper Chloride, CuCl, Hexagonal, Copper Fluoride, CuF, Copper Iodide, CuI, Cubic (Marshite), Copper Iodide, CuI, Hexagonal, Silver Bromide, AgBr (Bromirite), Silver Iodide, AgI, (Iodargirite), Silver Iodide, AgI, (Miersite), Actinium Bromide, AcBr₃, Actinium Chloride, AcCl₃, Actinium Fluoride, AcF₃, Actinium Iodide, AcI₃, Aluminum Bromide, AlBr₃, Aluminum Chloride, AlCl₃, Aluminum Fluoride, AlF₃, Aluminum Iodide, AlI₃, Americium (III) Bromide, AmBr₃, Americium (III) Chloride, AmCl₃, Americium (III) Fluoride, AmF₃, Americium (III) Iodide, AmI₃, Americium (IV) Fluoride, AmF₄, Antimony (III) Bromide, SbBr₃, Antimony (III) Chloride, SbCl₃, Zirconia, ZrO2, Zirconium Oxide Ceramic, Zirconia MgO Stabilized, Zirconium Oxide Ceramic—Zirconia, Y203 Stabilized, Zirconium Oxide Ceramic Zirconia, Tetragonal, Y203 Stabilized, Ceraflex 3Y Thin Zirconia Ceramic, Yttria Stabilized, Ceraflex 8Y Thin Zirconia Ceramic Oxygen Ion Conductor, Yttria Stabilized, Yttrium-Stabilized Zirconia (YSZ) Single Crystal Substrate, AREMCO 516 Ultra-temp, AREMCO Cerama-cast™ 583, AREMCO Cerama-cast™ 646, AREMCO Pyro-Paint™ 634-ZO, CeramTec Grade 950 Toughened Alumina (Al₂O₃—ZrO₂), CeramTec Grade 965 Toughened Alumina (Al₂O₃—ZrO₂), CeramTec Grade 848 Zirconia (ZrO₂), Channel Industries 5400 Lead Zirconate Titanate Piezoelectric, Channel Industries 5500 Lead Zirconate Titanate Piezoelectric, Channel Industries 5600 Lead Zirconate Titanate Piezoelectric, AREMCO Pyro-Putty® 653, AREMCO Pyro-Putty® 1000, AREMCO Pyro-Putty® 2400, AREMCO Pyro-Putty® 2500, Barium Boride, BaB6, Calcium Boride, Cerium Boride, CeB6, GE Advanced Ceramics AC6043 Titanium Diboride/Boron Nitride Composite, GE Advanced Ceramics Titanium Diboride/Boron Nitride Composite Vacuum Metallizing Boats, GE Advanced Ceramics HCT-30 Titanium Diboride (TiB₂) Powder, GE Advanced Ceramics HCT-40 Titanium Diboride (TiB₂) Powder, GE Advanced Ceramics HCT-30D Titanium Diboride (TiB₂) Powder, GE Advanced Ceramics HCT-F Titanium Diboride (TiB₂) Powder, GE Advanced Ceramics HCT-S Titanium Diboride (TiB₂) Powder, Ceralloy 225, Titanium Diboride, TiB₂ and other commercially available ceramics.

The motive device 18 is for moving the surface 16 with respect to the source 22, or with respect to an area of the surface where the solution is initially applied. The motive device can move the source of the solution with respect to the surface, the surface with respect to the source, or both. The movement can be rotational, reciprocating in a vertical or horizontal direction, opposed lateral or vertical edges of the surface moving reciprocatingly up and down with respect to one another (i.e., in a direction generally perpendicular to the surface), torsional, undulating, or any combination of these movements.

In FIG. 12, the motive device 18 has a drive motor 27 and a shaft 28 for moving the surface with respect to the source of solution. The drive motor 27 is capable of producing uniform rotational speeds at low RPM. The motor 27 has controls (not shown) for adjusting or selecting the speed of rotation (RPM) and the time period of the rotation for entering a programmed series of rotations or direction of rotation (i.e., clockwise, counterclockwise or alternating between these two directions) or the like.

The gas delivery device or system 20 has a source of gas 40 supplying a gas manifold 42 for distributing a flow of gas from the source in a controlled fashion over the surface 16 using a gas controller 44. The source of gas 40 includes a liquid nitrogen vaporizer 46 that converts liquid nitrogen to gaseous nitrogen. A fluid pathway 48 conveys the gas from the vaporizer 46, through the controller 44, and to the manifold 42.

The manifold 42 can take on many forms, depending on whether the surface 16 is positioned on an internal or external surface. FIG. 12 shows the surface 16 on an internal surface of the cylinder 26, and FIG. 13 shows the surface 16 positioned on an outer surface of the cylinder 26. The manifold 42 shown in FIG. 12 is a tube 50 having a plurality of perforations 52. The tube can be a rigid, semi-rigid, or flexible.

FIG. 13 shows an embodiment of the manifold 42 for conveying gas to the external surface 16′ and includes a perforated plenum of a flexible, rigid, or semi-rigid material and, in a preferred form of the invention, has a hemicylindrical outer portion that generally follows the curvature of the outer surface 16′.

The motor 27 is mounted to a support frame 56 having a vertical riser 58, which, in a preferred form of the apparatus, can be adjusted to an angle α, with respect to a horizontal surface such as a floor. In a preferred form of the apparatus, the angle a will be from 20 degrees to 160 degrees, more preferably from 45 degrees to 135 degrees, even more preferably from 75 degrees to 115 degrees, and most preferably from 80 to 100 degrees (or any range or combination of ranges therein).

A second cylinder 30 is mounted to the shaft 28 by a flange and defines a sleeve that is dimensioned to coaxially receive the first cylinder 26. The second cylinder can be fabricated from any of the materials described herein that are suitable for the first cylinder. In a preferred form, the second cylinder is fabricated from a polymeric material such as a COC, a polyester, a polycarbonate, a polyolefin, a polystyrene, or a substituted or unsubstituted acrylic acid, methacrylic acid, or ethyacrylic acid containing polymers. A most preferred form of the apparatus is a poly(methyl methacrylate) or PMMA, sold under the trade name Plexiglas.

The apparatus is capable of making particles described above in a batch mode (FIG. 12) or in a continuous mode (FIG. 13). For batch mode processing, a quantity of the three component solution is added to an interior of the first cylinder 26. The first cylinder 26 is leveled using the stand 56, such that the liquid level of the solution is about the same level from the top to the bottom of the glass vessel (a glass lip at the mouth of the vessel prevents the solution from running out). The motor 27 then rotates the second cylinder 26 and the glass vessel 26 for several seconds, until a uniform coating 19 forms on the internal surface 16 of the vessel. In a controlled manner, nitrogen gas is then permitted to flow into the manifold 42 such that the perforations 52 in the manifold distributes the gas uniformly over the surface of the thin layer 19 of solution coating the glass.

This causes a laminar flow of nitrogen gas to flow over the liquid surface, reducing boundary layer effects and promoting efficient evaporation without heating. There may be some beneficial turbulent mixing of the nitrogen at the surface of the thin film that facilitates evaporation, but the net effect is laminar flow of the gas over the surface of the thin film and out of an open end of the vessel. The nitrogen gas, containing solvent vapors, exits a mouth of the vessel and is vented into a hood or appropriate exhaust or solvent recovery system.

Within a short period, such as one minute, the thin layer of solution 19 turns opaque and, then, dries to a hazy film on the surface of the glass. The nitrogen is allowed to flow at a reduced rate for several minutes in order to completely dry the resulting small spherical particles. Then the gas flow is stopped, and the nitrogen manifold removed. Samples may then be easily obtained from anywhere in the vessel. The vessel can then be tilted back and the microspheres washed to the bottom of the vessel where they are easily collected.

For a continuous operation, FIG. 13 shows the solution 22 in a holding tank and being continuously applied to the surface 16′ by the roller 24. A pressure washer 60 sprays the dried film with a cleaning fluid, such as water, and a removal device 62 that continuously removes particles by engaging the surface with a member having a smooth blade such as a squeegee, scraper or smooth blade knife.

FIG. 14 shows a portion of another apparatus for continuously forming particles and includes the surface 16′, moveable in the directions indicated by the arrows. The surface is carried by a conveyor belt. The conveyor belt is trained about drive rollers 66. One or both of the drive rollers are connected to a motive source such as a motor described above having motor controls. The apparatus includes the roller 24, which applies the solution to the surface 16′. The conveyor is activated to move the surface to cause a thin film of the solution to form. The film is then exposed to the gas, washed, and removed by squeegee 62. The conveyor apparatus can be modified to have the solution applied and removed from the same side of the conveyor belt.

### EXAMPLES

All active agents were purchased from Spectrum, Chemicals & Laboratory Products, unless specified otherwise.

### Example 1

Small Spherical Particles of Beclomethasone Dipropionate (BDP)

Micronized beclomethasone dipropionate (BDP) USP was weighed and dissolved in ethanol USP to form a 10 mg/ml BDP-ethanol solution. 1.2 ml of the BDP-ethanol solution was mixed with 0.8 ml of deionized water to form a 3:2 vol/vol BDP-ethanol/water solution. The solution was transferred to a 1000 ml round Pyrex® flask of a modified rotary evaporator (modified Rotavapor-R complete, Buchi), and rotated in the flask for a few seconds to form a thin film on the inner surface of the flask. After the thin film was established, a controlled pure nitrogen inflow was allowed to enter the flask at a controlled 65-75 LPM flow rate. As the liquid phase evaporated, the solubility of the drug in the remaining mixed solvent rapidly decreased and a phase separation took place. Precipitation of the drug molecule was observed, as it formed a translucent layer on the surface of the flask. After the drug precipitated, the flask's rotation and nitrogen inflow were continued for several minutes to assure complete evaporation of the liquid phase and dryness of the small spherical particles. The resulting small spherical particles were collected by resuspending them in a small quantity of ice-cold deionized water and sonicating the suspension to facilitate the separation of the small spherical particles from the inner surface of the flask. The final steps were flash-freezing and lyophilization.

Particle morphology for the following examples was obtained using Scanning Electron Microscopy (SEM, FEI Quanta 200, Hilsboro, Oreg.). The sample was prepared for analysis by placing a small amount on carbon double-stick tape fixed to an aluminum sample mount. The sample was then sputter-coated using a Cressington sputter coater 108 Auto for 90 seconds and 20 mA. A second SEM instrument (Amray 1000, Bedford, Mass.) was used to obtain additional images of the small spherical particles, due to its higher resolution capabilities.

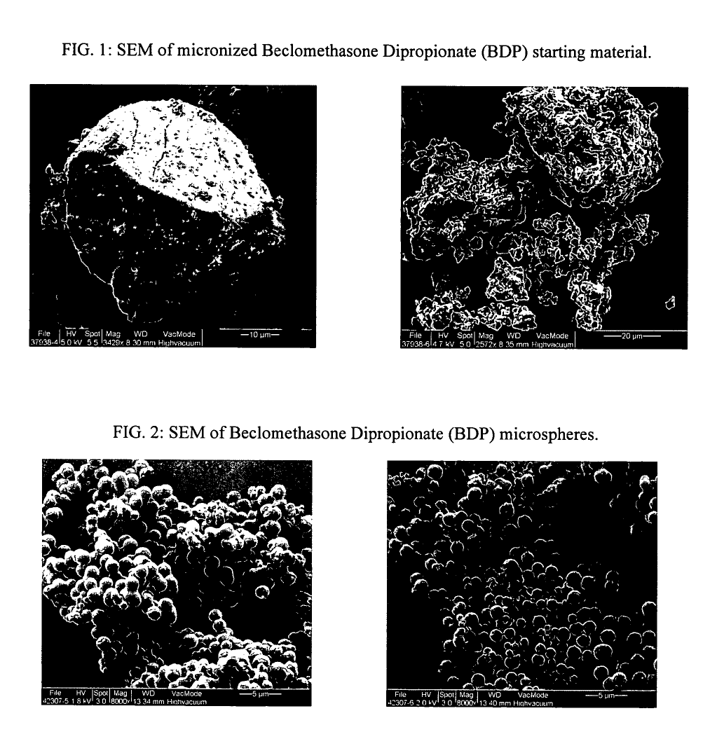

FIG. 1 presents SEM micrographs of the micronized BDP starting material. FIG. 2 presents micrographs of the resulting BDP small spherical particles. The micronized BDP starting material vary in shape and size and have a broad particle size distribution of 5-50 microns, while some of the particles are larger than 50 microns (FIG. 1). In contrast, the BDP small spherical particles have a uniform spherical shape, have a narrow particle size distribution and have an average diameter of about 1-2 microns. The small spherical particles have smooth surfaces compared to the rough surface of the micronized starting material (FIG. 2).

X-Ray Powder Diffraction (XRPD) measurements were performed on the BDP starting material (BDP#1) and on two batches of BDP small spherical particles (BDP#2 and BDPJM0710) to examine the degree of crystallinity of the starting material and to compare it with the crystallinity of BDP small spherical particles. The XRPD patterns were obtained by using an X-ray powder diffractometer (Shimadzu XRD-6000) with a rotating anode. The powders were scanned over a 2θ range by a continuous scan at 3°/min (0.4 sec/0.02° step) from 2.5 to 40 degrees, using Cu K α radiation. Diffracted radiation was detected by a Nal scintillation detector and analyzed using XRD-6000 v. 4.1.

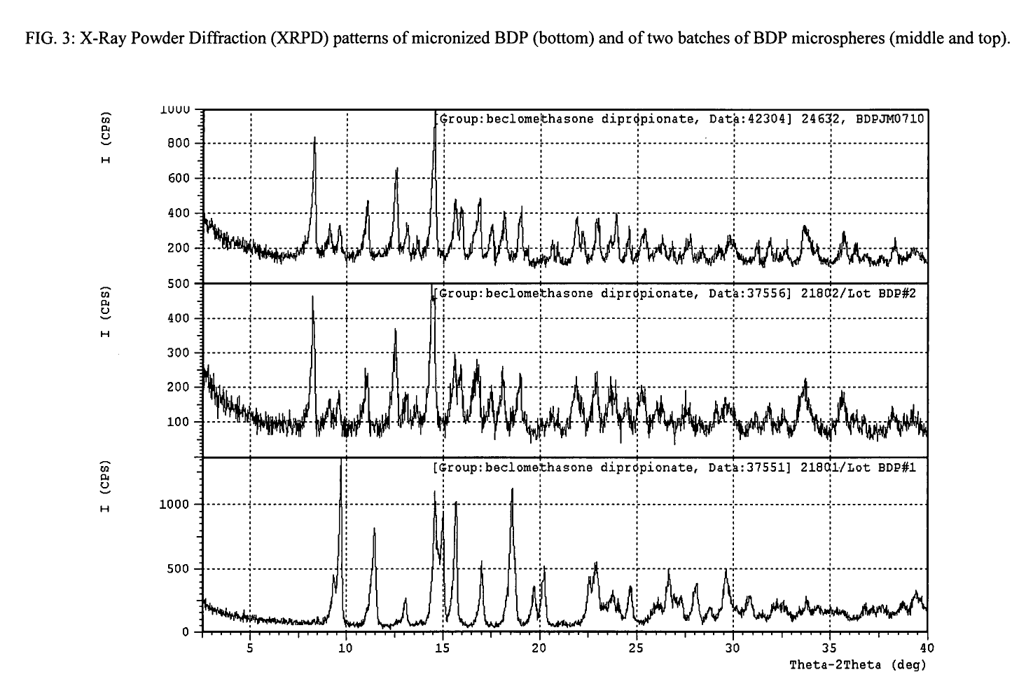

The XRPD pattern for the BDP starting material (FIG. 3, bottom) displays resolution of reflections, indicating the sample is crystalline. The XRPD patterns of the two BDP microsphere batches (FIG. 3, middle and top) also display resolution of reflections, indicating crystalline samples. However, the XRPD patterns for the small spherical particle samples are different from the XRPD patterns of the micronized starting material in terms of peak positions in 2θ, suggesting the samples are composed of different forms or mixtures of forms than the starting material. The two lots of small spherical particles showed identical peaks, which suggest that these independent batches that were prepared according to the method described above are homogeneous small spherical particles in terms of their degree of crystallinity. In addition, it shows that the process is reproducible.

### Example 2

Small Spherical Particles of Budesonide

Micronized budesonide USP was weighed and dissolved in ethanol USP to form 10 mg/ml budesonide-ethanol solution. 1.2 ml of the budesonide-ethanol solution was mixed with 0.8 ml of deionized water, to form a 3:2 vol/vol budesonide-ethanol/water solution. The solution was transferred to a 1000 ml round Pyrex® flask of a modified rotary evaporator (modified Rotavapor-R complete, Buchi), and the process continued as described in Example 1 for small spherical particles comprising BDP.

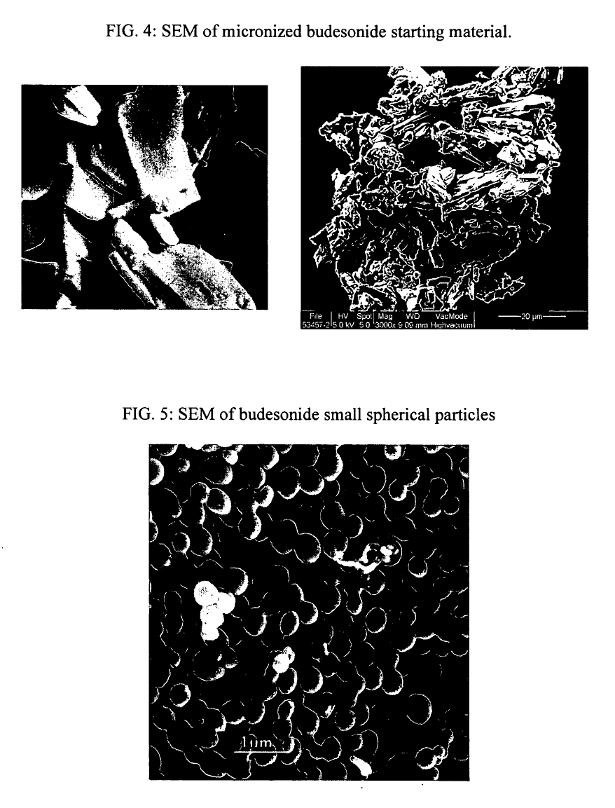

Particle morphology for the following examples was obtained using Scanning Electron Microscopy (FEI Quanta 200, Hilsboro, Oreg.). FIG. 4 presents SEM of micronized budesonide starting material and FIG. 5 presents SEM of the resulting budesonide small spherical particles.

Similar to Example 1, micronized budesonide starting material varies in shape and size and has a broad size distribution of 5-100 microns. Some of the particles are larger than 100 microns (FIG. 4). On the contrary, the budesonide small spherical particles have uniform spherical shape, have a narrow size distribution and are 1-2 microns in average size. (FIG. 5).

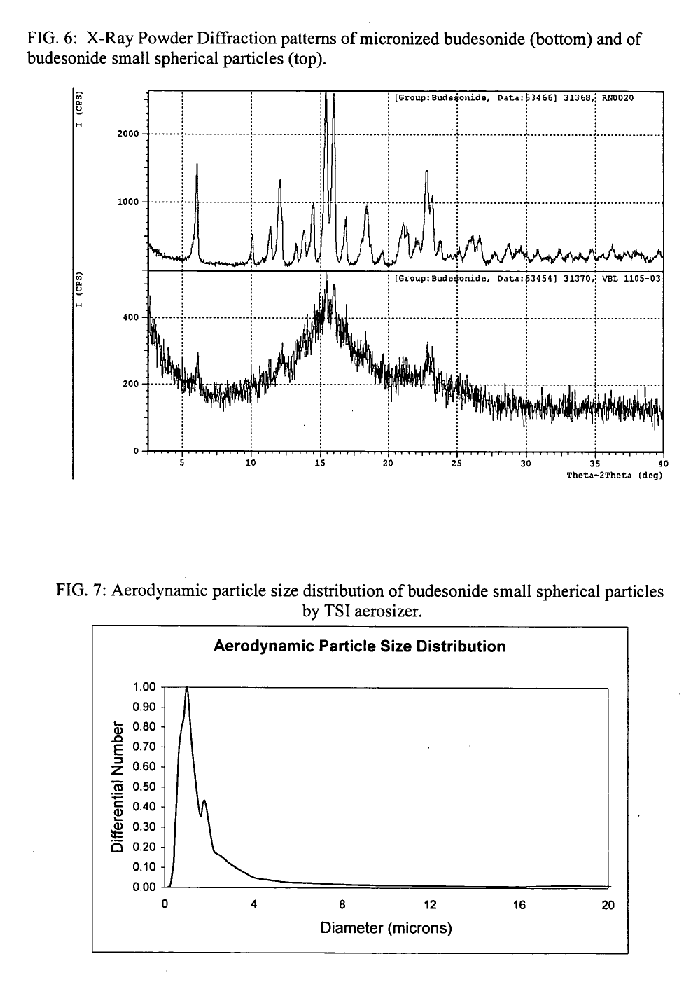

XRPD measurements were performed on the budesonide starting material (RN0020) and on a batch of budesonide small spherical particles to examine the degree of crystallinity of the starting material and to compare it with the crystallinity of budesonide small spherical particles (FIG. 6). The XRPD pattern of budesonide starting material showed distinctive peaks, characteristic of the crystalline state. In contrast, the XRPD of the budesonide small spherical particles was continuous and typical of the non-crystalline or amorphous state.

Aerodynamic particle size distribution was measured by a time-of-flight method, using a TSI Corporation Aerosizer (TSI, St. Paul, Minn.). FIG. 7 shows a narrow aerodynamic particle size distribution with 90% of the particles less than 2.4 microns.

### Example 3

Small Spherical Particles of Itraconazole

Micronized itraconazole USP (Wycoff, Inc.) was weighed and a volume of acetone USP was added to form a 10 mg/ml itraconazole-acetone suspension. The suspension was formed in a glass vial with a screw cap to prevent the rapid evaporation of acetone. The sealed vial was vortexed and then inserted into a water bath preheated to 70° C. The vial was left in the bath for 5-10 minutes, which allowed the dissolution of the itraconazole and the formation of an itraconazole-acetone solution. The vial was removed from the 70° C. bath and was left to cool to room temperature. After cooling, 2.48 ml of the itraconazole-acetone solution was mixed with 1.52 ml of a 10% ethanol in deionized water solution to form a 62% itraconazole-acetone/38% water-ethanol vol/vol solution. The total volume of the itraconazole-acetone/water-ethanol solution was 4 ml. The solution was transferred to a 1000 ml round Pyrex® flask of a modified rotary evaporator (modified Rotavapor-R complete, Buchi), and the process continued as described in Example 1 for small spherical particles of BDP.

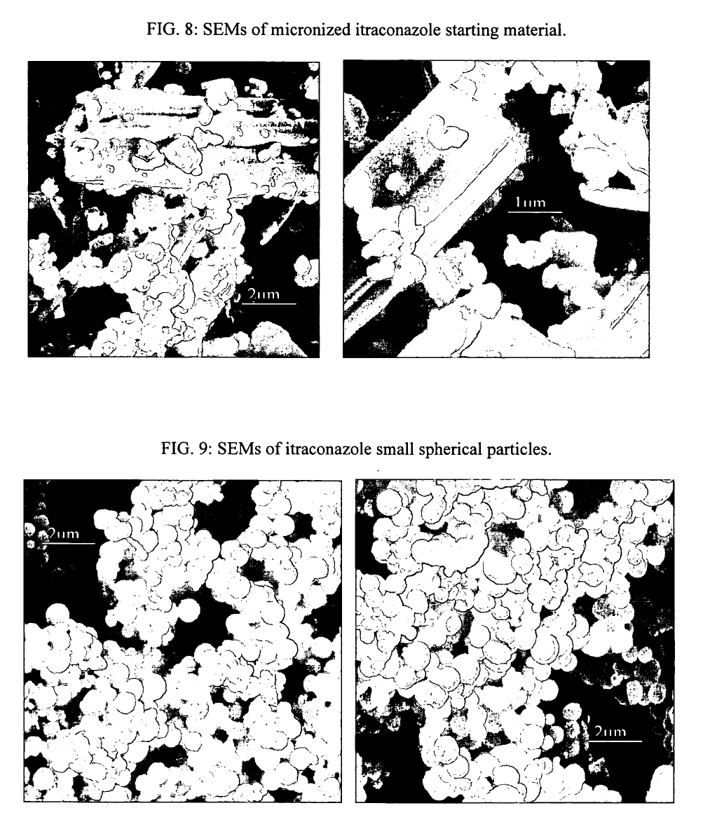

FIG. 8 presents SEMs of micronized itraconazole starting material and FIG. 9 presents SEMs of the resulting itraconazole small spherical particles. Micronized itraconazole starting material varies in shape and size and has a broad particle size distribution of 0.1-20 microns. Some of the particles are larger than 20 microns (FIG. 8). In contrast, the itraconazole small spherical particles have a uniform spherical shape, a narrow particle size distribution and an average diameter of 0.5-2 microns. (FIG. 9).

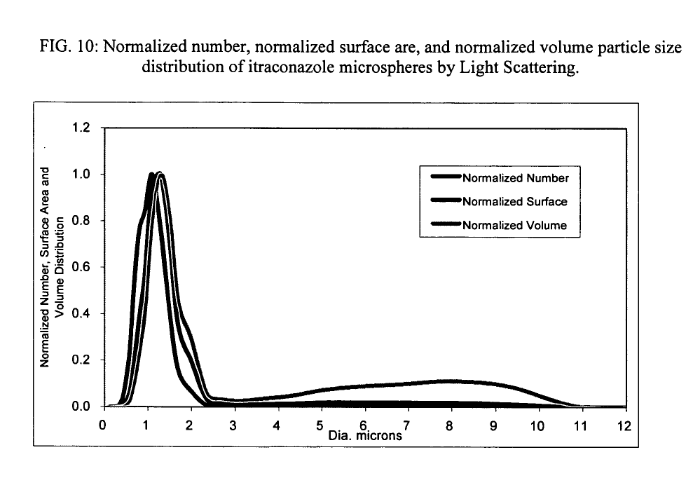

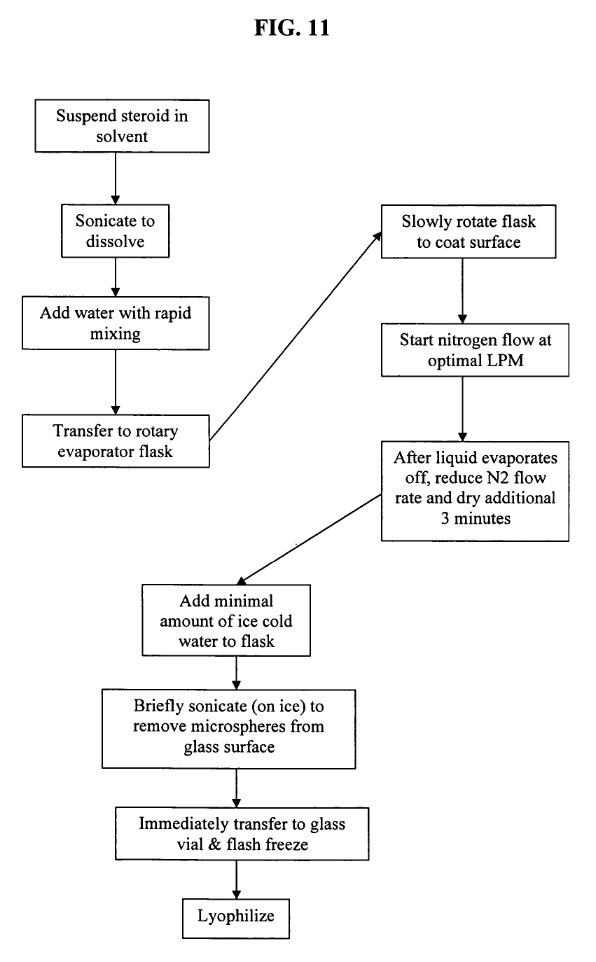

Particle size distribution was measured by light scattering using a Coulter instrument (Beckman Coulter LS 230, Miami, Fla.). Normalized number, normalized surface area, and normalized volume size distribution of itraconazole small spherical particles are presented in FIG. 10. The three normalized distributions overlap, which demonstrates the monodispersity of the particles. It also shows that the microparticles are homogeneously distributed in aqueous solution in the presence of surfactant, and that they do not tend to agglomerate.

### Example 4

Small Spherical Particles of Estradiol

Micronized estradiol USP (Akzo Nobel) was weighed and inserted into a screw cap glass tube. Ethanol USP was added to the tube to form a 5 mg/ml estradiol in ethanol solution.

Small spherical particles of estradiol were formed by two methods. In the first method, a drop of the estradiol-ethanol solution was placed on a glass slide, and ambient air was blown on the slide until dryness. As the ethanol evaporated from the drop, the estradiol precipitated out of solution and formed a translucent film on the slide. The slide was left on the laboratory bench for an additional 20 minutes to allow complete evaporation of the ethanol.

In the second method, a drop of the solution was placed on a glass slide that rested on a bed of ice. The slide was covered with aluminum foil to prevent wetting. Ambient air was blown on the slide until dryness. As the ethanol evaporated from the drop, the estradiol precipitated out of solution and formed a translucent film on the slide. The slide was left on 20 the bed of ice for additional 20 minutes to allow complete evaporation of the ethanol.

The slides were examined under light microscope to verify the existence of small spherical particles and to estimate the size distribution of the resulting estradiol small spherical particles. Small spherical particles were formed on both slides, the one left at ambient air temperature and the one that was placed on the ice bath.

### Example 5

Small Spherical Particles of Fludrocortisone

Micronized fludrocortisone USP was weighed and inserted into a screw cap glass tube. Ethanol USP was added to the tube to form a 5 mg/ml fludrocortisone in ethanol solution.

Small spherical particles of fludrocortisone were formed by two methods. In the first method, a drop of the fludrocortisone-ethanol solution was placed on a glass slide, and ambient air was blown on the slide until dryness. As the ethanol evaporated from the drop, the fludrocortisone precipitated out of solution and formed a translucent film on the slide. The slide was left on the laboratory bench for an additional 20 minutes to allow complete evaporation of the ethanol.

In the second method, a drop of the fludrocortisone-ethanol solution was placed on a glass slide that rested on a bed of ice. The slide was covered with aluminum foil to prevent wetting. Ambient air was blown on the slide until dryness. A translucent film of fludrocortisone was formed on the slide. The slide was left on the bed of ice for additional 20 minutes to allow complete evaporation of the ethanol.

The slides were examined under a light microscope to verify the existence of small spherical particles and to estimate the size distribution of the resulting fludrocortisone small spherical particles. Small spherical particles were formed on both slides, the one left at ambient air temperature and the one that was placed on the an ice bath.

### Example 6

Small Spherical Particles of Flucinonide

Micronized flucinonide USP was weighed and inserted into a screw cap glass tube. A relative volume of ethanol USP was added to the tube to form a 5 mg/ml flucinonide in ethanol suspension. The tube with the suspension was inserted into a thermal bath, preheated to 45° C. Part of the flucinonide did not dissolve at that elevated temperature, however, additional heating was avoided.

A drop of the flucinonide-ethanol suspension was placed on a glass slide. Ambient air was blown on the slide until complete dryness. A translucent film of flucinonide was formed on the slide. The slide was left on the laboratory bench for an additional 20 minutes to allow complete evaporation of the ethanol.

The slide was examined under a light microscope to verify the existence of small spherical particles and to estimate the size distribution of the resulting flucinonide small spherical particles. The resulting flucinonide small spherical particles had a uniform particle size distribution and an average diameter of 1-1.15 microns.

### Example 7

Effect of Various First And Second Solvents On Steroid Small Spherical Particle Formation

The ability to form small spherical particles of two steroids, beclomethasone dipropionate (BDP) and fluticasone propionate (FP) was examined in matrix experiments using acetone, ethanol, methanol, and methyl ethyl ketone (MEK) individually as the first solvents and water and heptane individually as the second solvents. The amount of second solvent added to the first solvent/steroid solution was varied as 0%, 10%, 20%, 30%, and 40% (v/v). MEK is not miscible with water and methanol is not miscible with heptane, so those combinations were not included in the experiment.

Either BDP or FP was weighed into a large screw cap glass tube and the solvent of choice was added (w/v) to yield a final concentration of 2 mg/mL. The tubes were vortexed and sonicated to completely dissolve the steroid. The sealed tubes containing these solutions were used as stock solutions for subsequent mixture with the appropriate second solvent. Immediately prior to use, an appropriate amount of the second solvent was slowly added to the first solvent/steroid solution while mixing to avoid premature precipitation. After adding the second solvent, the solutions were visually examined to ensure that premature precipitation had not occurred.

A fixture was constructed such that a 0.125-inch diameter orifice nozzle was positioned 1.75 inches above a standard glass microscope slide. Nitrogen gas was allowed to flow at 5 liters per minute through the nozzle and over the slide such that the flow direction of the gas was perpendicular to the surface of the slide. One or two drops of the test solution were placed on the slide directly under the orifice, and the nitrogen flow continued until the slide was dry (one to three minutes depending on the solution composition). Each slide was then examined under a polarized light microscope (Leica EPISTAR, Buffalo, N.Y.) using incident lighting. Each slide was graded for the presence of predominantly small spherical particles (+), a mixture of small spherical particles and non-spherical particles (+/−), and predominantly non-spherical particles (−). Variations in size and size distribution were observed between different test solutions. The results are tabulated below.

TABLE 1
Formation of small spherical particles of Beclomethasone dipropionate

| | Second solvent | | | | | | | | |
|---|---|---|---|---|---|---|---|---|---|
| | 0% | | | | | | | | |
| | (2 mg/mL | | | | | | | | |
| First | stock | 10% | 20% | 30% | 40% | 10% | 20% | 30% | 40% |
| solvent | solution) | water | water | water | water | heptane | heptane | heptane | heptane |
| acetone | + | + | + | + | + | + | + | +/− | +/− |
| ethanol | + | + | + | + | + | + | +/− | +/− | +/− |
| methanol | + | + | + | + | + | N/A | N/A | N/A | N/A |
| MEK | +/− | N/A | N/A | N/A | N/A | + | + | + | + |

TABLE 2
Formation of small spherical particles of Fluticasone propionate

| | Second solvent | | | | | | | | |
|---|---|---|---|---|---|---|---|---|---|
| | 0% | | | | | | | | |
| | (2 mg/mL | | | | | | | | |
| First | stock | 10% | 20% | 30% | 40% | 10% | 20% | 30% | 40% |
| solvent | solution) | water | water | water | water | heptane | heptane | heptane | heptane |
| acetone | + | + | +/− | − | − | + | + | +/− | +/− |
| ethanol | +/− | +/− | +/− | − | − | − | − | − | − |
| methanol | − | − | ppt | ppt | ppt | N/A | N/A | N/A | N/A |
| MEK | +/− | N/A | N/A | N/A | N/A | − | − | − | − |

Legend:
(+) = predominantly small spherical particles;
(+/−) = a mixture of small spherical particles and non-spherical particles;
(−) = predominantly non-spherical particles,
N/A = test not performed due to non-miscible solvents; and
ppt = the steroid precipitated out during addition of the second solvent.

Although water was not added to the 0% concentrations, some water would have been absorbed from the air during the experiment. However, the amount of water absorbed by the solvent is assumed to be well under 10%. The results indicate that the ability to form small spherical particles varies according to: 1) the organic small molecule used, 2) the first solvent composition, 3) the second solvent composition, and 4) the amount of second solvent in the final formulation. Also notable is the fact that various first solvents and second solvents other than water can be used to create small spherical particles by this method. In this case the alkane heptane was substituted for water as the second solvent and successfully used to fabricate small spherical particles of BDP and FP.

### Example 8

Evaporative Cooling During Formation of BDP Small Spherical Particles

BDP small spherical particles were fabricated on glass slides by the same method as Example 7 using acetone as the first solvent and water as the second solvent, except that the flow rate of nitrogen gas was 2.5 liters per minute. As the solvent evaporated, the temperature of the droplet on the slide was measured using a non-contact infrared sensor (Cole-Parmer, Vernon Hills, Ill., Model # A39671-22). The time interval between placing the drop on the slide under the nitrogen gas flow and the lowest temperature recorded was noted. Samples containing 10% water and 40% water (v/v) were compared.

The temperature measured on the surface of the dry slide with nitrogen flow was a constant 21.8° C. measured for several minutes before the start of each experimental run. Therefore, the nitrogen gas itself was not changing temperature during the test period. As the 10% water/acetone/BDP solution evaporated, the temperature dropped from 21.8° C. to 9.6° C. in 7 seconds. A repeat run resulted in a temperature drop to 9.8° C. in 7 seconds, so the test method was reproducible. In contrast, as the 40% water/acetone/BDP solution evaporated, the temperature dropped from 21.8° C. to 11.6° C. in 12 seconds. The reduction in the amount of the temperature drop and the increased time to reach the coolest temperature can be explained by the decreased amount of acetone in the 40% water sample.

Small spherical particles of BDP were observed using the light microscope (described in Example 7) on all of the glass slides, where the 40% water yielded a uniform size distribution of particles estimated at 1 to 2 micrometer diameter. In contrast, the 10% water slides yielded a broader size distribution of microspheres estimated at 0.5 to 10 micrometers in diameter. These results indicate that evaporative cooling does occur using this method to fabricate BDP microspheres and different cooling rates and absolute temperature changes are associated with different size distributions of small spherical particles.

While specific embodiments have been illustrated and described, additional modifications may be envisioned without departing from the spirit of the invention and the scope of protection is only limited by the scope of the accompanying claims.

1. Small spherical particles, comprising an organic molecule with a molecular weight of less than 1500 Daltons, with a narrow particle size distribution, wherein the organic molecule is at least 70% and less than or equal to 100% by weight of the particle.

2. The particles of claim 1 wherein the organic molecule is 90% or greater by weight of the particle.

3. The particles of claim 1 wherein the organic molecule is 95% or greater by weight of the particle.

4. The particles of claim 1 having an average particle size from about 0.01 μm to about 200 μm.

5. The particles of claim 1 having an average particle size of from about 0.1 μm to about 10 μm.

6. The particles of claim 1 having an average particle size of from about 0.1 μm to about 5 μm.

7. The particles of claim 1 wherein the organic molecule is hydrophobic.

8. The particles of claim 1 wherein the organic molecule is hydrophilic.

9. The particles of claim 1 wherein the organic molecule is sparingly water-soluble.

10. The particles of claim 1 wherein the organic molecule has a solubility in water of less than 10 mg/mL.

11. The particles of claim 1 wherein the organic molecule has a solubility in water of less than 1 mg/mL.

12. The particles of claim 1 wherein the active agent is selected from the group consisting of pharmaceutically therapeutic agents, diagnostic agents, cosmetics, nutritional supplements, and pesticides.

13. The particles of claim 12 wherein the pharmaceutically therapeutic agent is selected from the group consisting of: steroids, beta-agonists, antifungal and anti-microbial agents, bacteriastatic agents, taxanes, amino acids, aliphatic compounds, aromatic compounds, and urea compounds.

14. The particles of claim 13 wherein the steroid is selected from the group consisting of: beclomethasone, budesonide, fluticasone, flunisolide, fluocinolone, betamethasone, mometasone, ciclesonide, prednisolone, prednisone, hydrocortisone, dexamethasone, triamcinolone, momethasone, and pharmaceutically accepted salts, esters, hydrates and solvates of these compounds.

15. The particles of claim 13 wherein the beta-agonist is a short-acting beta adrenergic agonist or a long-acting beta-adrenergic agonist.

16. The particles of claim 15 wherein the short-acting beta adrenergic is selected from the group consisting of: salbutamol, pirbuterol, metaproterenol, terbutaline and fenoterol.

17. The particles of claim 15 wherein the long-acting beta-adrenergic is selected from the group consisting of: salmeterol, formoterol, bambuterol, clenbuterol, procaterol, bitoleterol, broxaterol and tulobuterol, and pharmaceutically accepted salts, esters, hydrates and solvates of these compounds.

18. The particles of claim 1 wherein the small spherical particles contain a combination of two or more active agents.

19. The particles of claim 1 further comprising a bulking agent.

20. The particles of claim 13 wherein the anti-fungal agent is selected from the group consisting of: itraconazole, fluconazole, posaconazole.

21. The particles of claim 1 having a density greater than 0.5/cm^{3.}

22. The particles of claim 1 having a density greater than 0.75/cm³.

23. The particles of claim 1 having a density greater than 0.85/cm³.

24. The particles of claim 1 having a density from about 0.5 to about 2 g/cm³.

25. The particles of claim 1 having a density from about 0.75 to about 1.75 g/cm³.

26. The particles of claim 1 having a density from about 0.85 g/cm³ to about 1.5 g/cm³.

27. The particles of claim 1 wherein the organic molecule further comprises a polymorph or pseudo-polymorph of the organic molecule.

28. The particles of claim 1 wherein the particles are crystalline, semi-crystalline or non-crystalline.

29. The particles of claim 1, where the particles are modified to result in controlled release of the organic molecule.

30. The particles of claim 1, wherein the particles are suitable for routes of administration selected from the group consisiting of parenteral, topical, oral, rectal, nasal, pulmonary, vaginal, buccal, sublingual, transdermal, transmucosal, ocular, transocular, and otic.

31. The particles of claim 1, wherein the particles are suitable for pulmonary delivery.

32. The particles of claim 31 wherein pulmonary delivery includes delivery to upper airways of the lung, the middle airways of the lung and/or to the periphery of the lung.

33. The particles of claim 1 wherein the particles are suitable for oral delivery to the gastrointestinal tract.

34. The particles of claim 1, wherein the particles are suitable for delivery by a device selected from the group consisting of a dry powder inhaler, a metered dose inhaler, and a nebulizer.

35. The particles of claim 1, wherein the small spherical particles are suitable for local treatment or systemic treatment.

36. The particles of claim 1, wherein the particles are suitable for transdermal delivery.

37. The particles of claim 1, wherein the particles are suitable for intravenous delivery, intramuscular delivery, or subcutaneous delivery.

38. A method for preparing small spherical particles of a low molecular weight organic moleculeactive agent, the method comprising the steps of:
preparing a solution of the active agent in a first solvent, the active agent having solubility in the first solvent;
adding a second solvent to the solution to form a three component solution of the two solvents and the active agent, wherein the solubility of the active agent in the second solvent is lower than in the first solvent;
spreading the solution on a surface to form a thin film of the solution on the surface; and
evaporating the solvents from the solution to form small spherical particles of the active agent on the surface by passing a stream of gas over the film to form small spherical particles coating on the surface, wherein the gas does not react with the active agent.

39. The method of claim 38 further comprising the step of removing the small spherical particles from the surface.

40. The method of claim 39 wherein the removal step comprises the step of adding a third solvent to the surface.

41. The method of claim 39 wherein the third solvent is a single solvent or a mixture of solvents.

42. The method of claim 41 wherein the third solvent is the same as the first solvent or the second solvent.

43. The method of claim 41 wherein the third solvent is the same as the second solvent.

44. The method of claim 3 further comprising the step of removing the second solvent to form dry powder of the small spherical particles.

45. The method of claim 38 wherein the step of preparation of the solution of the active agent in the first solvent is by adding the agent to the first solvent and sonicating the mixture to dissolve the agent in the first solvent.

46. The method of claim 38 wherein the surface is a material selected from a polymer, metal, ceramic, or glass.

47. The method of claim 38 wherein the surface is a glass surface.

48. The method of claim 38 wherein the surface is a polymer selected from the group consisting of: polyolefins, cyclic olefins, bridged polycyclic hydrocarbons, polyamides, polyesters, polyethers, polyimides, polycarbonates, polystyrene, polyvinyl chloride, ABS, polytetrafluoroethylene (PTFE), styrene and hydrocarbon copolymers, and synthetic rubbers.

49. The method of claim 38 wherein the surface is a metal selected from the group consisting of: aluminum, stainless steel, vanadium, platinum, titanium, gold, beryllium, copper, molybdenum, osmium, nickel or other suitable alloys or metals or metal composites.

50. The method of claim 38 wherein the surface is a ceramic.

51. The method of claim 50 wherein the ceramic is a metal oxide.

52. The method of claim 38 wherein the material can be rigid, semi-rigid or flexible.

53. The method of claim 38 wherein the step of spreading the mixture on a surface further comprises the step of moving the surface.

54. The method of claim 53 wherein the surface is moved in a manner selected from the group consisting of rotational, reciprocating, opposed lateral or vertical edges of the surface moving reciprocatingly up and down with respect to one another torsional, undulating or a combination of these movements.

55. The method of claim 38 wherein the surface has a smooth or a textured surface.

56. The method of claim 38 wherein the surface has a cross-sectional shape selected from the group consisting of: flat, curved, undulating or irregular.

57. The method of claim 38 wherein the step of spreading the solution on a surface to form a thin film comprises the step of transferring the solution to a rotary evaporating flask and slowly rotating the flask to form a coating of the solution on the inner surface of the flask.

58. The method of claim 38 wherein the gas is selected from the group consisting of: nitrogen, hydrogen, helium, and argon.

59. The method of claim 38 wherein the gas is nitrogen.

60. The method of claim 38 further comprising the step of continuing the gas inflow at a reduced flow rate after small spherical particles formation initiated in order to dry the small spherical particles.

61. The method of claim 38 wherein the second solvent is cooled to a temperature that reduces the solubility of the active agent

62. The method of claim 39 wherein removing the small spherical particles from the surface further comprises the step of sonicating the solution.

63. The method of claim 62 wherein the step of sonicating is carried out on ice.

64. The method of claim 44 wherein the step of removing the second solvent comprises the step of lyophilizing.

65. The method of claim 38, wherein the first solvent is an organic solvent and is selected from the group consisting of: N-methyl-2-pyrrolidinone (N-methyl-2-pyrrolidone), 2-pyrrolidinone (2-pyrrolidone), 1,3-dimethyl-2-imidazolidinone (DMI), dimethylsulfoxide, dimethylacetamide, volatile ketones, acetone, methyl ethyl ketone, acetic acid, lactic acid,acetonitrile, methanol, ethanol, isopropanol, 3-pentanol, n-propanol, benzyl alcohol, glycerol, polyethylene glycol (PEG), PEG-4, PEG-8, PEG-9, PEG-12, PEG-14, PEG-16, PEG-120, PEG-75, PEG-150, polyethylene glycol esters, PEG-4 dilaurate, PEG-20 dilaurate, PEG-6 isostearate, PEG-8 palmitostearate, PEG-150 palmitostearate, polyethylene glycol sorbitans, PEG-20 sorbitan isostearate, polyethylene glycol monoalkyl ethers, PEG-3 dimethyl ether, PEG-4 dimethyl ether, polypropylene glycol (PPG), polypropylene alginate, PPG-10 butanediol, PPG-10 methyl glucose ether, PPG-20 methyl glucose ether, PPG-15 stearyl ether, propylene glycol dicaprylate/dicaprate, propylene glycol laurate, and glycofurol (tetrahydrofurfuryl alcohol polyethylene glycol ether), propane, butane, pentane, hexane, heptane, octane, nonane, decane or a combination thereof.

66. The method of claim 38 wherein the first solvent or the second solvent or both the first solvent and the second solvent are volatile.

67. The method of claim 38 wherein the first solvent is ethanol and the second solvent is water.

68. The method of claim 38 wherein the second solvent is an alkane selected from the group consisting of hexane, heptane, octane, nonane and decane.

69. The method of claim 38 wherein the steps are carried out at about 25° C. or below.

70. An apparatus for forming small spherical particles from a solution containing a low molecular weight agent comprising:
a surface mounted for movement;
a fluid delivery device for applying the solution to an area of the surface;
a motive device connected to the surface for moving the area with respect to the fluid delivery device; and
a gas plenum positioned proximate the surface for providing gas under pressure to the surface.

71. The apparatus of claim 70 wherein the surface has a cross-sectional shape selected from the group consisting of: flat, curved, undulating or irregular.

72. The apparatus of claim 70 wherein the cross-sectional shape of the surface is curved.

73. The apparatus of claim 72 wherein the motive device is a motor having a shaft for rotating the curved surface.

74. The apparatus of claim 73 wherein the curved surface is positioned on an outer surface or an inner surface of a cylinder.

75. The apparatus of claim 74 wherein the cylinder is made from a material selected from the group consisting of a polymer, metal, ceramic or glass.

76. The apparatus of claim 75 wherein the gas plenum has a length and has a plurality of perforations along the length.

77. The apparatus of claim 75 further comprising an applicator for applying the solution to the surface.

78. The apparatus of claim 77 wherein the applicator sprays the solution on the surface or applies it by direct contact with the surface.

79. The apparatus of claim 77 wherein the applicator is a roller having a first portion contacting the solution and a second portion contacting the surface.

80. The apparatus of claim 77 further comprising a squeegee for removing the film from the surface.
